(19) United States
(12) Patent Application Publication
(10) Pub. No.: US 2014/0121894 A1
(43) Pub. Date: May 1, 2014

(54) EMISSIONS MONITOR STOPLIGHT INTERFACE

(71) Applicants: Michael Drew, Dexter, MI (US); Brian Herron, Dexter, MI (US)

(72) Inventors: Michael Drew, Dexter, MI (US); Brian Herron, Dexter, MI (US)

(21) Appl. No.: 14/064,436

(22) Filed: Oct. 28, 2013

Related U.S. Application Data
(60) Provisional application No. 61/719,052, filed on Oct. 26, 2012.

(51) Int. Cl. G01N 33/00

(52) U.S. Cl. CPC G01N 33/0063
USPC 701/33.5

(57) ABSTRACT

A system includes a processor in communication with a port, wherein the port is configured to be connected to a port of a vehicle, the port being in communication with an electronic control unit. The system further includes a visual output device in communication with the processor. The processor being configured to read data from the electronic control unit, determine if the vehicle has (a) failed an emissions test, (b) the vehicle is running an emissions test, or (c) passed an emissions test. The processor is further configured to output to the visual output device if the vehicle has (a) failed an emissions test, (b) the vehicle is running an emissions test, or (c) passed an emissions test.

### CROSS REFERENCE TO RELATED PATENT APPLICATION

This application claims priority to U.S. Provisional Patent Application 61/719,052, which is hereby incorporated by reference in its entirety.

### BACKGROUND

1. Field of the Invention

The present invention generally relates vehicle emissions interface.

2. Description of Related Art

From 1996-present, vehicles have onboard diagnostics which allow technicians and emissions test stations to evaluate the vehicle's condition. The vehicle's ECU was designed from the factory to continuously test its own emissions and only set the emissions monitors to “ready” once those tests have successfully completed. If the tests are not completed, or if they have failed, the monitor status is reported as “not ready”.

When cars are brought in for state emissions, they are tested by a state-authorized facility that plugs a scantool into the car's OBD2 port. The scantool checks the ECU to see if any trouble codes are present, and if all of the vehicle's emission monitors have passed. If all of the monitors have passed and no trouble codes are present, the vehicle passes the states emissions test. If any trouble codes are present or if any of the monitors have not completed their tests, the vehicle fails the state emissions test. In many states, this emissions test is required for annual or bi-annual registration.

A car can fail emissions testing for a number of reasons, but almost all of those reasons require the car owner to take their car to a repair shop. The repair shop will then use a scantool and connect to the car's OBD2 port to determine why the car did not pass the test. They will usually replace parts or adjust tune the vehicle up so it meets emissions and clear any trouble codes, which also resets the emission monitors to not ready.

The problem in today's model after the vehicle has been repaired. Even though the vehicle has been repaired and is producing emissions within the satisfactory limits, the vehicle's onboard diagnostic system must run its own internal tests and set the emissions monitors to “ready” before the vehicle will pass the state emissions test. This process can take anywhere from 5 miles of driving to over 100 miles of driving. There is no indication to the vehicle owner that the internal tests are completed and the monitors have been set to “ready”.

If a vehicle is brought to the state-authorized test facility before the ECU has completed its own internal tests and set the monitors to “ready”, the vehicle will again fail the state emissions test. Sometimes a vehicle owner may take their car in several times after the repair and repeatedly fail the test because they do not know the vehicle has not completed its internal tests yet.

### SUMMARY

A system includes a processor in communication with a port, wherein the port is configured to be connected to a port of a vehicle, the port being in communication with an electronic control unit. The system further includes a visual output device in communication with the processor. The processor being configured to read data from the electronic control unit, determine if the vehicle has (a) failed an emissions test, (b) the vehicle is running an emissions test, or (c) passed an emissions test. The processor is further configured to output to the visual output device if the vehicle has (a) failed an emissions test, (b) the vehicle is running an emissions test, or (c) passed an emissions test.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 illustrates a vehicle and its basic electronic components;

FIG. 2 illustrates a system configured to determine the emission state of a vehicle; and

FIG. 3 is another embodiment of the system of FIG. 2, wherein this embodiment contains a display for displaying emissions related information.

### DETAILED DESCRIPTION

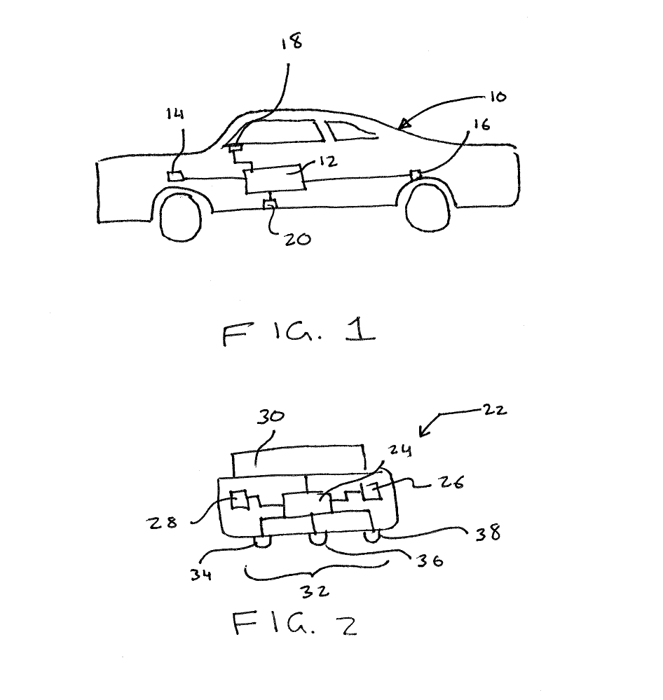

Referring now to FIG. 1, a vehicle 10 is shown. The vehicle 10 may include an Electronic Control Unit (“ECU”) 12. The ECU may be a single controller or may be multiple controllers that are intended to monitor or modify one or more subsystems of the vehicle. For example, the ECU 12 may monitor or modify engine control subsystems, transmission subsystems, safety subsystems, emissions systems or any other subsystems commonly found within the vehicle 10.

The vehicle 10 may be one of any number of different vehicles. The vehicle 10 may be a common passenger car, truck, heavy duty truck, construction equipment, boat, and/or airplane. The vehicle 10 can essentially be any type of vehicle capable of transporting items and/or people from one point to another. In communication with the ECU 12 are sensors 14 and 16. The sensors 14 and 16 may be a single sensor or may be multiple groups of sensors configured to monitor one or more of the subsystems of an automobile. For example, the sensor 14 may be an engine control and/or powertrain control sensor that monitors engine and power train variables of the vehicle 10. These variables may include things such as throttle position, RPM, shift position, emissions systems or other engine and power train related information. The information collected by sensor 14 is then relayed to the ECU 12.

The ECU 12 may also be connected to other sensors as well, such as sensor 16. Sensor 16 may be dedicated to other vehicle subsystems, such as tire pressure monitoring, wheel speed, safety systems, emissions systems or any other type of vehicle subsystem. Information collected by the ECU 12 from the sensors 14 and/or 16 can be related to a display device 18 which may be located on the dashboard of an automobile. In addition, the display device 18 may be an external device as capable of displaying information collected from the sensors 14 and/or 16 to the driver of the vehicle 10.

The ECU 12 of the vehicle 10 is further in communication of the port 20. The port 20 allows external devices to communicate with the ECU 12 of the vehicle 10. These external devices can receive information from the ECU 12 that are originated with the sensors 14 and/or 16. The port 20 may be any type of port but may be an OBD2 port. As it is well known, any one of a number of different devices can be connected to the port 20 either directly or using a pass through device, such as a J2534 pass through device.

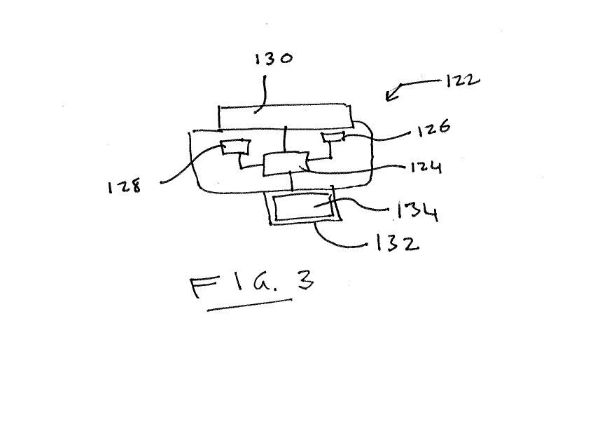

Referring to FIG. 2, the system 22 is shown. As its primary components the system 22 includes a processor 24 that may be in communication with one or more memory units 26 and 28. Of course, the memory units 26 and 28 may be incorporated within the processor 24 or may be incorporated together separate from the processor 24. Additionally, it should be understood, that additional memory units may be shown as well. The system 22 also includes a port 30. The port 30 may be any kind of port, but it's preferably a port that is configured to mate with the port 20 of the vehicle 10 of FIG. 1. As such, the port 30 may also be an OBD2 style port.

The system 22 may include one or more display devices 32. In this embodiment, the display devices include a green light emitting diode (“LED”) 34, a yellow LED 36, and a red LED 38. The display devices 32 are each separately in communication with the controller 24 of the system 22.

Referring to FIG. 3, another embodiment of the system 122 is shown. Like reference numerals have been utilized to refer to like components, with the exception that the number “1” is placed before these reference numerals. The description of the previously described elements will not be given. The system 122 differs from the system 22 of FIG. 2 in that the system 122 has a display device 132 with a display area 134.

The display device 132 may be a small LCD type display device or other display device capable of generating information within a display area 134. Any number of different display devices may be shown. The display device may display letters and/or numbers or may further display symbols, including colors. For example, while the system 22 of FIG. 2 may light up one of the colored LED's 34, 36, and 38, the display area 134 may simply just display the color red, yellow, or green. Of course, any one of a number of different colors can be utilized as well.

Referring to FIGS. 2 and 3, the controllers 24 and 124 may execute software stored either on the controller 24 or 124 or on the memory devices 26, 126, 28, or 128. The software stored on these devices configures the process to perform a number of different tasks. Here, the software configures the processor 24 or 124 to read data from the electronic control unit 12 of the vehicle 10 and determine the vehicle as either (a) failed an emissions test, (b) the vehicle is running an emissions test, (c) passed an emissions test. From there, the processor 24 and 124 is configured to output to the display devices 32 or 132 if the vehicle has either (a) failed an emissions test, (b) the vehicle is running an emissions test, or (c) passed an emissions test.

Information may be relayed to the display device such that, for example, the green LED 34 is lit when the vehicle has passed an emissions test. The yellow LED 36 may be lit if the vehicle is running an emissions test, or the red LED 38 may be lit when the vehicle has failed an emissions test. Similarly, the display device 132 of FIG. 2 may show on the display are 134 letters or numbers indicating if the vehicle has failed an emissions test, is running an emissions test, or passed the emissions test. Like before, colors may also be used to indicate whether the vehicle has passed a test, failed a test, or is running a test. The colors may match the colors lit on the LED's described in FIG. 2.

The processor may be further configured to include or exclude specific readiness monitors or pass fail analysis to allow custom configuration for different state inspection or maintenance requirements.

The system 22 or 122 is configured to operate over a number of different product protocols. The system can be configured to operate over a number of different protocols. The number of protocols that the system can operate under includes, but is not limited to, the CAN protocol, K-Line/ISO9141/ISO14230 protocol, J1850 VPW, or J1850PWM. Further, the system can employ an encrypted boot loader that allows for secure update of firmware over the above-listed different protocols. Of course, any which one of a number of protocols can be utilized and should not be limited to the ones just specifically listed above. Further, the system may employ an encrypted boot loader that allows secure update of firmware over whatever protocol is utilized.

The system also has the ability to lock to a single vehicle and may further include a storage device in communication with the processor, wherein the storage device is configured to store vehicle data internally for later retrieval and process.

The system 22 or 122 also has the ability to go into a low power mode. The low power mode can be determined via SAE J1979 Mode 1 PID 0 request that the electronic control unit is no longer powered on. The system may also be able to determine the protocol used by the vehicle by using the SAE J1699-3 protocol determining and then store this information internally so that the time consuming protocol determination only needs to be done once, at power on, when the system 22 or 122 is connected to the vehicle 10. Finally, the system 22 or 122 will not wake from a low power sleep mode if the battery voltage falls below a configured low limit. The system 22 or 122 may also be able to determine if the system 22 or 122 should enter a sleep mode based on the running status of the vehicle 10.

The system 22 or 122 may store information retrieved from the ECU 12 and the memory units 24, 124, 26, or 126 of the systems 22 or 122. Information stored can later be retrieved for later analysis.

As a person skilled in the art will readily appreciate, the above description is meant as an illustration of implementation of the principles this invention. This description is not intended to limit the scope or application of this invention in that the invention is susceptible to modification, variation and change, without departing from the spirit of this invention, as defined in the following claims.

1. A system comprising,
a processor in communication with a port, wherein the port is configured to be connected to a port of a vehicle, the port being in communication with an electronic control unit;
a visual output device in communication with the processor;
the processor being configured to read data from the electronic control unit, determine if the vehicle has (a) failed an emissions test, (b) the vehicle is running an emissions test, or (c) passed an emissions test; and
the processor being configured to output to the visual output device if the vehicle has (a) failed an emissions test, (b) the vehicle is running an emissions test, or (c) passed an emissions test.

2. The system of claim 1, wherein the visual output device comprises three separate lights.

3. The system of claim 2, wherein the processor is further configured to provide power to one of the lights for each situation if the vehicle has (a) failed an emissions test, (b) the vehicle is running an emissions test, or (c) passed an emissions test.

4. The system of claim 3, wherein the processor is further configured to provide power to (a) a green light if the vehicle passed the emissions test, (b) a yellow light if the vehicle is running the emissions test, and (c) a red light if the vehicle failed an emissions test.

5. The system of claim 1, wherein the processor is further configured to include or exclude specific readiness monitors for pass/fail analysis to allow custom configuration for differing State inspection and maintenance requirements.

6. The system of claim 1, wherein the processor is further configured to include or exclude specific diagnostic codes for pass/fail analysis to allow custom configuration for differing State inspection and maintenance requirements.

7. The system of claim 1, wherein the system is configured to operate over the CAN protocol.

8. The system of claim 1, wherein the system is configured to operate over the K-Line/ISO9141/ISO14230 protocol.

9. The system of claim 1, wherein the system is configured to operate over the J1850 VPW protocol.

10. The system of claim 1, wherein the system is configured to operate over the J1850PWM protocol.

11. The system of claim 1, wherein the system employs an encrypted bootloader that allows for secure update of firmware over CAN protocol.

12. The system of claim 1, wherein the system employs an encrypted bootloader that allows for secure update of firmware over K-Line/ISO9141/ISO14230 protocol.

13. The system of claim 1, wherein the system employs an encrypted bootloader that allows for secure update of firmware over J1850 VPW protocol.

14. The system of claim 1, wherein the system employs a bootloader that allows for secure update of firmware over J1850PWM protocol.

15. The system of claim 1, wherein the system has the ability to lock to a single vehicle.

16. The system of claim 1, further comprising a storage device in communication with the processor, wherein the storage device is configured to store vehicle data internally for later retrieval and analysis.

17. The system of claim 1, wherein the system is configured to go into low power mode upon determining via SAE J1979 Mode 1 PID 0 request that the electronic control unit is no longer powered on.

18. The system of claim 1, wherein the system is configured to go into low power mode (sleep) upon determining via SAE J1979 Mode 1 PID 0 request that the electronic control unit is no longer powered on.

19. The system of claim 1, wherein the system is configured to go into low power mode (sleep) upon determining via SAE J1979 Mode 1 PID 0x0C that engine is no longer running.

20. The system of claim 1, wherein the system is configured to wake from low power mode and attempts to determine if engine is running or electronic control unit is powered.

21. The system of claim 1, wherein the system determines the protocol used by vehicle using SAE J1699-3 protocol determination and stores this information internally so that the time consuming protocol determination only needs to be done once, at power on, when device is connected to vehicle.

22. The system of claim 1, wherein the system will not wake from a low power mode if the battery voltage falls below a configured low limit.
